(19) United States
(12) Patent Application Publication
(10) Pub. No.: US 2005/0079235 A1
(43) Pub. Date: Apr. 14, 2005

(54) Use of a polyphenol for the treatment of actinic keratosis

(76) Inventor: Eggert Stockfleth, Berlin (DE)

Correspondence Address: CLARK & ELBING LLP, 101 FEDERAL STREET, BOSTON, MA, 02110, US

(21) Appl. No.: 10/682,612

(22) Filed: Oct. 9, 2003

(51) Int. Cl. A61K 35/78; A61K 31/7048; A61K 31/353

(52) U.S. Cl. 424/729; 514/456; 514/27

(57) ABSTRACT

The present invention refers to a method for treating actinic keratosis by administering a pharmaceutically effective amount of a polyphenol to a patient as well as to the production of a medicament thereto.

### BACKGROUND OF THE INVENTION

The present invention refers to a method for treating actinic keratosis by administering a pharmaceutically effective amount of a polyphenol to a patient as well as to the production of a medicament thereto.

Skin cancer is a disease in which malignant (cancer) cells are formed in the tissues of the skin. The skin is the body's largest organ. It protects against heat, sunlight, injury, and infection, helps to control the body temperature and stores water, fat, and vitamin D. The skin has several layers, but the two main layers are the epidermis (upper or outer layer) and the dermis (lower or inner layer). Skin cancer usually starts growing in the epidermis, which is made up of three kinds of cells. The squamous cells are thin, flat cells that form the top layer of the epidermis. The basal cells are round cells below the squamous cells and melanocytes are found in the lower part of the epidermis. These cells produce melanin, the pigment that is responsible for the natural color of the skin. When skin is exposed to the sun, melanocytes are induced to produce more pigment causing the skin to tan or darken.

Skin cancer can occur anywhere but it is most common in skin that has been exposed to sunlight, such as the face, ears, neck, bald scalp, hands, shoulders, arms and/or the back. There are several types of cancer that start in the skin. The most common types are basal cell carcinoma and squamous cell carcinoma which are non melanoma skin cancers. Actinic keratosis is a skin condition that sometimes develops into squamous cell carcinoma.

Squamous cell carcinoma (SCC), the second most common skin cancer after basal cell carcinoma, afflicts more than 200,000 Americans each year. It arises from the epidermis and resembles the squamous cells that comprise most of the upper layers of skin. Squamous cell cancers may occur on all areas of the body including the mucous membranes, but are most common in areas exposed to the sun. Although squamous cell carcinomas usually remain confined to the epidermis for some time, they eventually penetrate the underlying tissues if not treated. In a small percentage of cases they spread (metastasize) to distant tissues and organs which can be fatal for the person afflicted. Metastasing squamous cell carcinomas most often arise on sites of chronic inflammatory skin conditions or on the mucous membranes or lips. Chronic exposure to sunlight causes most cases of squamous cell carcinoma because tumors appear most frequently on sun-exposed parts of the body. The rim of the ear and the lower lip are especially vulnerable to the development of these cancers. Squamous cell carcinomas may also occur where skin has suffered certain kinds of injury such as burns, scars, long-standing sores, sites previously exposed to X-rays and/or certain chemicals such as arsenic and petroleum by-products. In addition, chronic skin inflammation or medical conditions that suppress the immune system over an extended period of time may encourage development of squamous cell carcinoma. Occasionally, squamous cell carcinoma arises spontaneously on what appears to be normal, healthy or undamaged skin. Some researchers believe that a tendency to develop this cancer may be inherited.

Certain precursor conditions, some of which result from extensive sun damage, are sometimes associated with the later development of squamous cell carcinoma. They include actinic keratoses, actinic cheilitis, leukoplakia and Bowen's disease.

Actinic keratosis (AK), also known as a solar keratosis, arises on the skin surface. AK appears as rough, scaly crusty and/or slightly raised growths that range in color from brown to red and may be up to one inch in diameter. It appears most often in older people. The base may be light or dark, tan, pink, red or a combination of these or has the same color as the skin itself. The scale or crust is horny, dry and rough and is often recognized by touch rather than sight. Occasionally, it itches or produces a pricking or tender sensation. It can also become inflamed and surrounded by redness. In rare instances, actinic keratoses can even bleed. The skin abnormality or lesion develops slowly and generally reaches a size from an eighth to a quarter of an inch. Early on, it may disappear only to reappear later. Several Aks can often been seen at a time and are most likely to appear on the parts of the body most often exposed to sunshine. The growths may be flat and pink or raised and rough. AK can be the first step in the development of skin cancer. It is thus a precursor of cancer or a precancer. If treated early, almost all AKs can be eliminated without becoming skin cancers. But untreated, about two to five percent of these lesions may progress to squamous cell carcinomas. In fact, some scientists now believe that AK is the earliest form of Squamous Cell Carcinoma (SCC). These cancers are usually not life-threatening, provided they are detected and treated in the early stages. However, if this is not done, they can grow large and invade the surrounding tissues and, on rare occasions, metastasize or spread to the internal organs.

Chronic sun exposure is the cause of almost all AKs. Sun damage to the skin accumulates over time, so that even a brief exposure adds to the lifetime total. The likelihood of developing AK is highest in regions near the equator. However, regardless of climate, everyone is exposed to the sun. About 80 percent of solar UV rays can pass through clouds. These rays can also bounce off sand, snow and other reflective surfaces giving you extra exposure. AKs can also appear on skin that has been frequently exposed to artificial sources of UV light, such as tanning devices. More rarely, they may be caused by extensive exposure to X-rays or specific industrial chemicals. Individuals whose immune systems are weakened as a result of cancer chemotherapy, AIDS or organ transplantation are also at higher risk. AK is the most common type of precancerous skin lesion. Older people are more likely than younger ones to develop these lesions because cumulative sun exposure increases with the years. Some experts believe that the majority of people who live to the age of 80 will have AK.

The standard therapies might not be applicable in all patients, e.g. surgery in patients with severe concomitant diseases, or have severe side-effects and may result in skin breakdown, discoloration, irritation, damage to surrounding normal skin, swelling and/or scars.

Consequently, the problem underlying the present invention resides in providing an alternative therapy for at least most of the group of patients.

Surprisingly, it has been found that the treatment of the skin with at least one polyphenol, in particular with at least one catechin elicits a positive effect on actinic keratosis.

One subject-matter of the present invention is, therefore, a method for treating actinic keratosis by administering a pharmaceutically effective amount of a polyphenol or a mixture of polyphenols to a patient, in particular to a human.

The term “pharmaceutically effective amount” means an amount of at least one polyphenol which causes a positive effect on actinic keratosis, e.g. causes a reduction or disappearance of actinic keratosis, in particular with the aim to improve or cure actinic keratosis. Pharmaceutically effective amounts are e.g. formulations, preferably ointments, containing about 2% (w/w) to about 50% (w/w), especially about 5% (w/w) to about 20% (w/w), in particular about 10% (w/w) or about 15% (w/w) of at least one polyphenol or of a mixture of several (different) polyphenols. These amounts can be applied once or several times, e.g. 3 to 5 times a week for 6 to 12 weeks, until the positive effect on actinic keratosis occurs.

The term “about” means according to the invention a general error range of +/−20%, especially +/−10%, in particular +/−5%.

Polyphenols are naturally accurring phenolic compounds, preferably with 1, 2 or 3 aromatic rings, in particular with 2 aromatic rings, carrying at least two hydroxyl groups, such as catechols, flavons, flavonoids and/or anthocyanidins, e.g. pelargonidin, cyanidin, delphinidin, paonidin, petunidin, malvidin and/or hirsutidin, whereas catechols are naturally occurring polyphenols usually found in resins and/or lignins. Alternative names used in the literature for catechols are catechins, pyrocatechols or 1,2-dihydroxybenzenes.

The polyphenols, in particular the catechols employed in the present invention may be obtained either synthetically or from natural sources. The natural sources which may especially be mentioned are tea plants, in particular green tea. In this context, the natural constituents may be present in differing concentrations depending on the species and variety. In this connection, the polyphenols, in particular the catechols which are employed are preferably isolated or extracted from Camellia sinensis, Camellia asamica, Camellia bohea, Camellia chinensis and/or Camellia oleosa. All the components of tea plants, in particular the leaves, can be used for isolating or extracting the polyphenols, in particular the catechols. The polyphenols, in particular the catechols which are employed are preferably isolated from a tea extract, in particular from a green tea extract or easily extracted from a tea, in particular from a green tea. Suitable methods for the isolation or extraction of polyphenols, in particular catechols are described e.g. in U.S. Pat. No. 4,613,672, U.S. Pat. No. 4,673,530, U.S. Pat. No. 4,913,909, U.S. Pat. No. 6,096,359 or U.S. Pat. No. 4,248,789.

Generally, the polyphenols have the formula (I) 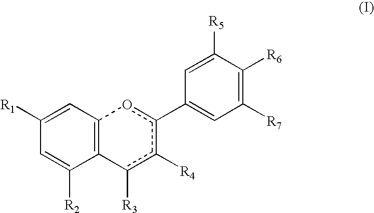
in which
R₁, R₂ and R₆ are independently from each other —H or —OH,
R₃ is —H or ═O,
R₄ is independently from each other —H, —OH or a group of the formula (III) 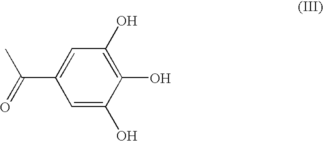
R₅ and R₇ are independently from each other —H, —OH or —OCH₃, and ----optionally represents a bond,
and the catechols have the formula (II) 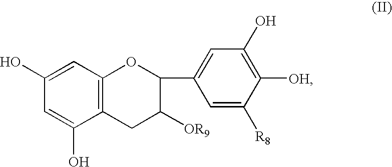
in which
R₈ is —H or —OH, and
R₉ is —H or a group of the formula (III)

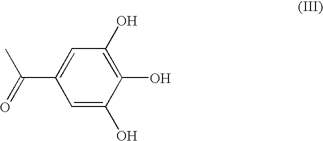

Examples of polyphenols are:

Polyphenol derivatives of flavan with the formula (IV):

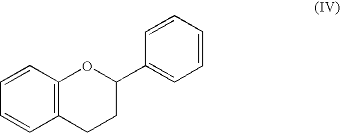

Polyphenol derivatives of flavan-3-ol with the formula (V):

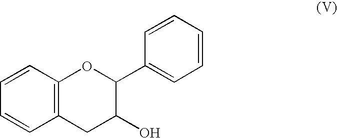

Polyphenol derivatives of flavanon with the formula (VI):

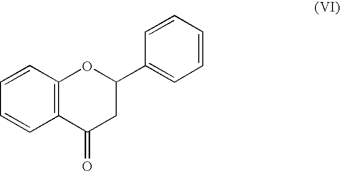

Polyphenol derivatives of flavon with the formula (VII):

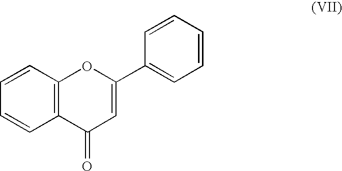

Polyphenol derivatives of flavonol with the formula (VIII):

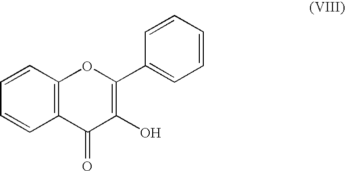

Polyphenol derivatives of chalcon with the formula (IX): 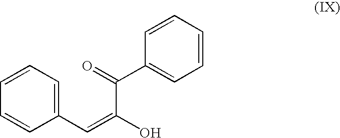
and anthocyanidins with the formula (X): 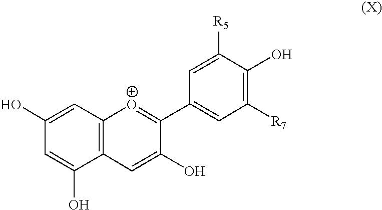
with R₅ and R₇ are independently from each other —H, —OH or —OCH₃, as e.g. in pelargonidin, cyanidin, delphinidin, paonidin, petunidin, malvidin or hirsutidin.

Preferably, the catechol is selected from catechol, catechol gallate, epicatechol, epicatechol gallate, epigallocatechol, epigallocatechol gallate, gallocatechol and/or gallocatechol gallate and in particular from (+)-catechol, (−)-catechol, (−)-catechol gallate, (+)-epicatechol, (−)-epicatechol, (−)-epicatechol gallate, (−)-epigallocatechol, (−)-epigallocatechol gallate, (+)-gallocatechol, (−)-gallocatechol and (−)-gallocatechol gallate.

The structural formula of the particular catechols is:

For (−)-epigallocatechol gallate (−)-EGCG:

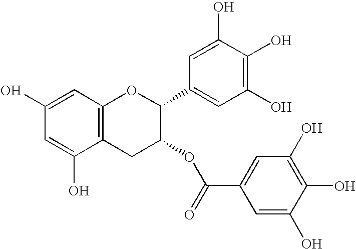

For (−)-epigallocatechol (−)-EGC:

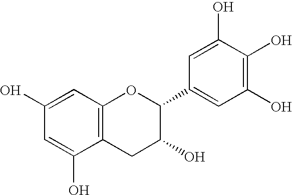

For (−)-epicatechol gallate (−)-ECG:

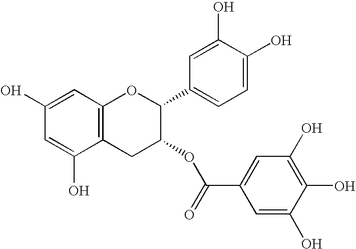

For (−)-epicatechol (−)-EC:

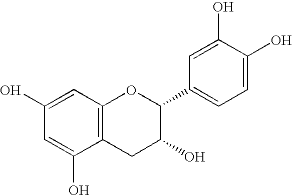

For (+)-epicatechol (+)-EC:

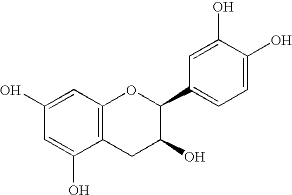

For (+)-catechol (+)-C:

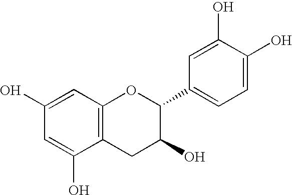

For (−) catechol (−)-C:

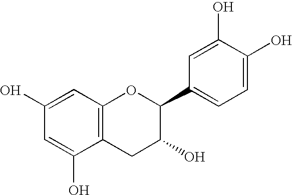

For (−)-gallocatechol gallate (−)-GCG:

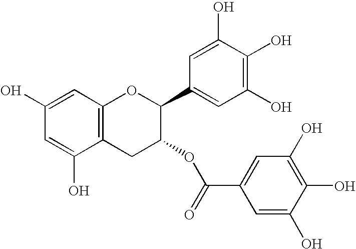

For (−)-catechol gallate (−)-CG:

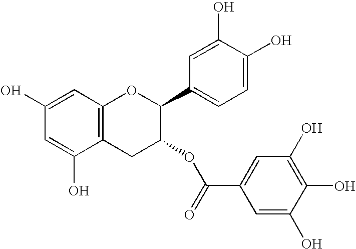

For (+)-gallocatechol (+)-GC:

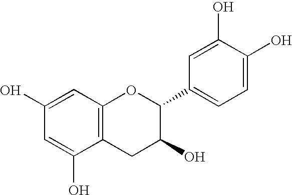

For (−)-gallocatechol (−)-GC:

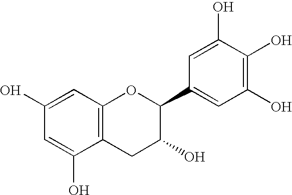

In another particularly preferred embodiment of the present invention the polyphenols, in particular the catechols, are present in the form of a mixture of polyphenols, in particular catechols, especially containing catechol, catechol gallate, epicatechol, epicatechol gallate, epigallocatechol, epigallocatechol gallate, gallocatechol and/or gallocatechol gallate, preferably in the stereochemistry as defined above.

Preferred catechols employed in the present invention are (−)-epicatechol, (−)-epicatechol gallate, (−)-epigallocatechol, (−)-epigallocatechol gallate, (+)-gallocatechol and/or (−)-gallocatechol gallate, in particular in form of a mixture containing about 2-20% (w/w) epicatechol, about 2-20% (w/w), epicatechol gallate, about 1-25% (w/w) epigallocatechol, about 40-75% (w/w) epigallocatechol gallate, about 0.05-5% (w/w) gallocatechol and/or about 0.5-20% (w/w) gallocatechol gallate, especially a mixture containing about 10.8% (w/w) of epicatechol, about 6.5% (w/w) of epicatechol gallate, about 9.2% (w/w) of epigallocatechol, about 54.8% (w/w) of epigallocatechol gallate and/or about 4.0% (w/w) of gallocatechol gallate, all of them preferably in the stereochemistry as defined above, in particular in form of a mixture containing about 10.8% (w/w) of (−)-epicatechol, about 6.5% (w/w) of (−)-epicatechol gallate, about 9.2% (w/w) of (−)-epigallocatechol, about 54.8% (w/w) of (−)-epigallocatechol gallate and/or about 4.0% (w/w) of (−)-gallocatechol gallate.

Alternatively, the mixture of catechols contains about 2-12% (w/w), preferably about 5-8% (w/w) epicatechol, about 4-15% (w/w), preferably about 5-7% (w/w), in particular about 5-6% (w/w) epicatechol gallate, about 1-8% (w/w), preferably about 2-3% (w/w), in particular about 6-8% (w/w) epigallocatechol, about 60-68% (w/w), preferably about 61-65% (w/w) epigallocatechol gallate, about 0.05-1% (w/w) gallocatechol and about 1-7% (w/w), preferably about 2-4% (w/w) gallocatechol gallate.

Consequently, the catechols can be used both individually and in the form of mixtures having different compositions as specified above. For example, a composition known under the tradename Polyphenon® 100 is composed of about 5.9% (w/w) of (−)-epicatechol, about 12.6% (w/w) of (−)-epicatechol gallate, about 17.6% (w/w) of (−)-epigallocatechol, about 53.9% (w/w) of (−)-epigallocatechol gallate and/or about 1.4% (w/w) of (−)-gallocatechol. As another example, a composition known under the tradename Polyphenon® E is composed of about 10.8% (w/w) of (−)-epicatechol, about 6.5% (w/w) of (−)-epicatechol gallate, about 9.2% (w/w) of (−)-epigallocatechol, about 54.8% (w/w) of (−)-epigallocatechol gallate and/or about 4.0% (w/w) of (−)-gallocatechol gallate.

The familiar methods of pharmaceutical technology are used, in a customary manner, for preparing pharmaceuticals which comprise one or more compounds according to the present invention and/or for using these pharmaceuticals in the application according to the invention. For this, the active compounds are worked up, together with one or more suitable, pharmaceutically acceptable additives, if necessary, into the medicinal forms which are suitable for the different indications and sites of administration. In this context, the pharmaceuticals can be prepared such that the rate of release in each case desired, for example a rapid accumulation and/or a delayed-release or depot effect, is achieved.

Consequently, another embodiment of the present invention is directed to the use of a pharmaceutical effective amount of a polyphenol, in particular a catechol or a mixture of (different) polyphenols, in particular catechols, as specified above, for the production of a medicament for the treatment of actinic keratosis, preferably for the topical administration of the polyphenol, in particular catechol, or polyphenol, in particular catechol mixture.

Examples of suitable additives are sodium alginate, as a gelatinizing agent for preparing a suitable base, or cellulose derivatives, such as guar or xanthan gum, inorganic gelatinizing agents, such as aluminum hydroxide or bentonites (what are termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. However, the organogels which are preferred are the hydrophobic organogels. Particular preference is given to hydrophobic additives, such as petroleum jelly, wax, oleyl alcohol, propylene glycol monostearate and/or propylene glycol monopalmitostearate, in particular isopropyl myristate. It is, of course, likewise possible to add skin-sedating and/or inflammation-inhibiting additives which are known to the skilled person, such as synthetically prepared active compounds and/or extracts and/or active compounds from medicinal plants, in particular bisobolol and panthenol. It is furthermore also possible to add dyes, for example yellow and/or red iron oxide and/or titanium dioxide for the purpose of matching as regards color.

Generally, the polyphenol, in particular the catechol or mixture of polyphenols, in particular catechols, is contained in a carrier, e.g. in the form of an emulsion, a gel, a cream or an ointment.

Customary emulsions, gels, creams and ointments of the mixed-phase or amphiphilic emulsion systems (oil/water-water/oil mixed phase), and also liposomes and transfersomes or plasters, preferably ointments and creams, particularly preferably an ointment, may be mentioned for conventional application to the skin. The catechol is preferably applied locally in the region in which there is actinic keratosis.

Emulsifiers which can be employed are anionic, cationic or neutral surfactants, for example alkali metal soaps, metal soaps, amine soaps, sulphurated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

It is possible to use vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats, for example based on, caprylic acid, capric acid, lauric acid or stearic acid, such as Softisan®, or triglyceride mixtures, such as Miglyol®, can be used as lipids, in the form of fatty and/or oleaginous and/or waxy components for preparing the ointments, creams or emulsions.

It is possible to use, for example, osmotically active acids and alkaline solutions, for example hydrochloric acid, citric acid, sodium hydroxide solution, potassium hydroxide solution, sodium hydrogen carbonate, and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions may be mentioned as additional forms which can be applied topically. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

The medicinal forms which are in each case suitable can be produced on the basis of pharmaceutico-physical principles in conformity with formulation guidelines and methods known to a skilled person.

As a further example, the pharmaceutical employed in the present invention preferably comprises about 35% (w/w) of isopropyl myristate, about 15% (w/w) of at least one catechol, about 24.5% (w/w) of petroleum jelly, about 20% (w/w) of wax, about 5% (w/w) of propylene glycol monostearate or propylene glycol monopalmitostearate and about 0.5% (w/w) of oleyl alcohol.

An alternative embodiment of the present invention is directed to a combination therapy.

Therefore, the present invention also encompasses a method for treating actinic keratosis by administering a pharmaceutically effective amount of a catechol or a mixture of catechols, as specified above, in combination with a different anticancer treatment and the preparation of a corresponding medicament. The administration of the different anticancer agent can be simultaneous with, prior to or after the administration of the polyphenol, in particular catechol or the mixture of polyphenols, in particular catechols.

According to the present invention the term “different anticancer treatment” refers preferably to surgery, electrodessication, curettage, excision, Mohs micrographic surgery, radiation, proton therapy, chemotherapy, photodynamic therapy, cryosurgery, laser, immunotherapy, vaccine therapy and/or biologic therapy. Preferred chemotherapeutic treatments encompass the use of podophyllin, 5-fluorouracil, bleomycin, interferon or imiquimod, and mixtures thereof. A preferred radiotherapy is X-ray radiation and/or y-radiation.

The following examples are intended to clarify the invention without restricting it. Skilled persons can modify the invention appropriately, within the bounds of customary ability, without departing from the protective scope.

### EXAMPLE 1

Patient: 65 years old, male with actinic keratoses known since 10 years;

The patient was treated with Polyphenon® E (15% ointment containing 35% (w/w) isopropyl myristate, 15% (w/w) catechol extract, 24.5% (w/w) petroleum jelly, 20% (w/w) wax, 5% (w/w) propylene glycol monostearate and 0.5% (w/w) oleyl alcohol):
Treated area: about 5 cm² on the forehead
Treatment schedule: 5 times a week (each with 10 hours)
Treatment period: 6 weeks

Treatment progression:
after about 13 days of treatment skin irritation of the treated area (more precisely treated areas afflicted by actinic keratoses) occurred
also an up-regulation of subclinical lesions occurred
skin irritation ameliorated during further treatment
after 12 weeks of treatment actinic keratoses have disappeared completely

### Example 2

Patient: 73 years old, male with actinic keratoses known since about 15 years, multiply pre-treated;

The patient was treated with Polyphenon® E (15% ointment containing 35% (w/w) isopropyl myristate, 15% (w/w) catechol extract, 24.5% (w/w) petroleum jelly, 20% (w/w) wax, 5% (w/w) propylene glycol monostearate and 0.5% (w/w) oleyl alcohol):
Treated area: about 5 cm² on the head
Treatment schedule: 3 times a week
Treatment period: 12 weeks

Treatment progression:
after 3.5 weeks of treatment skin irritation of the treated area afflicted by actinic keratoses occurred (but less intense than that of the patient of Example 1)>
after 12 weeks of treatment only single actinic keratoses have been left and after 16 weeks of treatment actinic keratoses have disappeared completely

1. A method for treating actinic keratosis by administering a pharmaceutically effective amount of a polyphenol to a patient.

2. The method according to claim 1, wherein the patient is a human.

3. The method according to claim 1, wherein the polyphenol is isolated from tea.

4. The method according to claim 1, wherein the polyphenol is extracted from a tea.

5. The method according to claim 9, wherein the tea is a green tea.

6. The method according to claim 1, wherein the polyphenol is isolated from a tea extract.

7. The method according to claim 1, wherein the polyphenol is selected from the group consisting of catechol, catechol gallate, epicatechol, epicatechol gallate, epigallocatechol, epigallocatechol gallate, gallocatechol and gallocatechol gallate.

8. The method according to claim 1, wherein the polyphenol has the general formula (I) 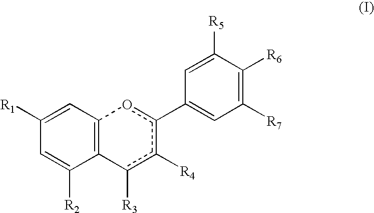
in which
R₁, R₂ and R₆ are independently from each other —H or —OH,
R₃ is —H or ═O and
R₄ is independently from each other —H, —OH or a group of the formula (III) 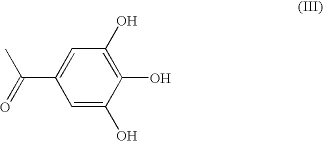
R₅ and R₇ are independently from each other —H, —OH or —OCH₃, and -----optionally represents a bond

9. The method according to claim 7, wherein the catechol has the general formula (II) 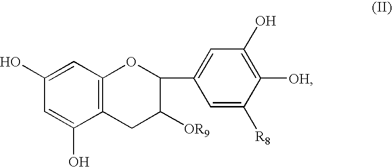
in which
R₈ is —H or —OH, and
R₉ is —H or a group of the formula (III)

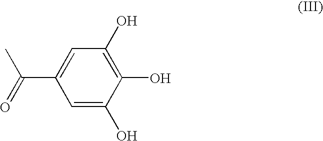

10. The method according to claim 7, wherein the catechol is selected from the group consisting of of (+)-catechol, (−)-catechol, (−)-catechol gallate, (+)-epicatechol, (−)-epicatechol, (−)-epicatechol gallate, (−)-epigallocatechol, (−)-epigallocatechol gallate, (+)-gallocatechol, (−)-gallocatechol and (−)-gallocatechol gallate.

11. The method according to claim 1, wherein the polyphenol is present in the form of a mixture of polyphenols.

12. The method according to claim 11, wherein the mixture of polyphenols is a tea extract.

13. The method according to claim 12, wherein the tea extract is a green tea extract.

14. The method according to claim 12, wherein the mixture of polyphenols is selected from the group consisting of catechol, catechol gallate, epicatechol, epicatechol gallate, epigallocatechol, epigallocatechol gallate, gallocatechol and gallocatechol gallate.

15. The method according to claim 14, wherein the catechols are selected from the group consisting of (−)-epicatechol, (−)-epicatechol gallate, (−)-epigallocatechol, (−)-epigallocatechol gallate, (+)-gallocatechol and (−)-gallocatechol gallate.

16. The method according to claim 14, wherein the catechols are selected from the group consisting of about 2-20% (w/w) epicatechol, about 2-20% (w/w) epicatechol gallate, about 1-25% (w/w) epigallocatechol, about 40-75% (w/w) epigallocatechol gallate, about 0.05-5% (w/w) gallocatechol and about 0.5-20% (w/w) gallocatechol gallate.

17. The method according to claim 14, wherein the catechols are selected from the group consisting of about 10.8% (w/w) of epicatechol, about 6.5% (w/w) of epicatechol gallate, about 9.2% (w/w) of epigallocatechol, about 54.8% (w/w) of epigallocatechol gallate and about 4.0% (w/w) of gallocatechol gallate.

18. The method according to claim 14, wherein the mixture contains about 10.8% (w/w) of (−)-epicatechol, about 6.5% (w/w) of (−)-epicatechol gallate, about 9.2% (w/w) of (−)-epigallocatechol, about 54.8% (w/w) of (−)-epigallocatechol gallate and about 4.0% (w/w) of (−)-gallocatechol gallate.

19. The method according to claim 14, wherein the catechols are selected from the group consisting of about 2-12% (w/w) epicatechol, about 4-15% (w/w) epicatechol gallate, about 1-8% (w/w) epigallocatechol, about 60-68% (w/w) epigallocatechol gallate, about 0.05-1% (w/w) gallocatechol and about 1-7% (w/w) gallocatechol gallate.

20. The method according to claim 14, wherein the catechols are selected from the group consisting of about 5-8% (w/w) epicatechol, about 5-7% (w/w) epicatechol gallate, about 2-3% (w/w) epigallocatechol, about 61-65% (w/w) epigallocatechol gallate and about 2-4% (w/w) of gallocatechol gallate.

21. The method according to claim 14, wherein the catechols are selected from the group consisting of about 5-8% (w/w) epicatechol, about 5-6% (w/w) epicatechol gallate, about 6-8% (w/w) epigallocatechol, about 61-65% (w/w) epigallocatechol gallate and about 2-4% (w/w) of gallocatechol gallate.

22. The method according to claim 1, wherein the polyphenol is combined with an additive.

23. The method according to claim 22, wherein the additive is selected from the group consisting of petroleum jelly, wax, oleyl alcohol, propylene glycol monostearate, propylene glycol monopalmitostearate and isopropyl myristate.

24. The method according to claim 1, wherein the polyphenol is administered topically.

25. The method according to claim 1, wherein the polyphenol is contained in a carrier selected from the group consisting of an emulsion, a gel, a cream and an ointment.

26. The method according to claim 1, wherein the method for treating actinic keratosis is combined with a different anticancer treatment.

27. The method according to claim 26, wherein the different anticancer treatment is selected from the group consisting of surgery, electrodessication, curettage, excision, Mohs micrographic surgery, radiation, proton therapy, chemotherapy, photodynamic therapy, cryosurgery, laser, immunotherapy, vaccine therapy and biologic therapy.

28. The method according to claim 27, wherein the chemotherapy is carried out with an agent selected from the group consisting of podophyllin, 5-fluorouracil, bleomycin, interferon, imiquimod, and mixtures thereof.

29. The method according to claim 27, wherein the radiation is selected from the group consisting of X-ray radiation and γ-radiation.
